(19) United States
(12) Patent Application Publication
(10) Pub. No.: US 2022/0309670 A1
(43) Pub. Date: Sep. 29, 2022

(54) METHOD AND SYSTEM FOR VISUALIZING INFORMATION ON GIGAPIXELS WHOLE SLIDE IMAGE

(71) Applicant: Applied Materials, Inc., Santa Clara, CA (US)

(72) Inventors: Sumit Jha, Bangalore (IN); Divakar Dass, Krishnagiri (IN); Nisarg Shah, Bengaluru (IN); Mayukh Bhattacharyya, Kolkata (IN); Suraj Rengarajan, Whitefield (IN)

(21) Appl. No.: 17/681,260

(22) Filed: Feb. 25, 2022

Related U.S. Application Data
(60) Provisional application No. 63/166,593, filed on Mar. 26, 2021.

(51) Int. Cl. G06T 7/00; G16H 30/40; G06T 7/11

(52) U.S. Cl. CPC G06T 7/0014; G16H 30/40; G06T 7/11; G06T 2207/10056; G06T 2207/30024; G06T 2207/20081; G06T 2200/24

(57) ABSTRACT

Methods and systems for visualizing information on gigapixels Whole Slide Image are described. In an example, a method for visualizing information includes providing an image viewer with a list of information to visualize, loading an image and a mask for an information source, and dynamically finding a zoom factor. If the zoom factor is not suitable for fine detailed view, then information for a coarse mask is shown. If the zoom factor is suitable for fine detailed view, then information for a fine detailed mask is chosen from a plurality of information sources.

### CROSS-REFERENCE TO RELATED APPLICATIONS

This application claims the benefit of U.S. Provisional Application No. 63/166,593, filed on Mar. 26, 2021 the entire contents of which are hereby incorporated by reference herein.

### BACKGROUND

### 1) Field

Embodiments of the present disclosure pertain to methods and systems for visualizing information on gigapixels Whole Slide Image.

### 2) Description of Related Art

A microscope can generate magnified images of a sample at any of a variety of magnification levels. The “magnification level” of an image refers to a measure of how large entities (e.g., cells) depicted in the image appear compared to their actual size. At higher magnification levels, a higher resolution image or a larger number of discrete images may be required to capture the same area of the sample as compared to a single image at a lower magnification level, thus requiring more space in a memory during storage.

Magnified images of a tissue sample can be analyzed by a pathologist to determine if portions (or all) or the tissue sample are abnormal (e.g., cancerous). A pathologist can analyze magnified images of a tissue sample by viewing portions of the tissue sample which appear to be abnormal at higher magnification levels.

### SUMMARY

Embodiments of the present disclosure include methods and systems for visualizing information on gigapixels Whole Slide Image.

In an embodiment, a method for visualizing information includes providing an image viewer with a list of information to visualize, loading an image and a mask for an information source, and dynamically finding a zoom factor. If the zoom factor is not suitable for fine detailed view, then information for a coarse mask is shown. If the zoom factor is suitable for fine detailed view, then information for a fine detailed mask is chosen from a plurality of information sources.

In an embodiment, a method for repeatedly training a machine learning model to segment magnified images of tissue samples, includes obtaining a magnified image of a tissue sample. In an embodiment, the method further comprises generating an automatic segmentation of the tissue sample using a machine learning model. In an embodiment, the method further comprises providing the automatic segmentation to a user through a user interface. In an embodiment, the method further comprises obtaining modifications to the automatic segmentation through the user interface. In an embodiment, the method further comprises determining an edited segmentation from the modifications. In an embodiment, the method further comprises determining updated values of model parameters based on the edited segmentation.

In an embodiment, a non-transitory computer readable storage medium having data stored representing software executable by a computer, the software including instructions for repeatedly training a machine learning model to segment magnified images of tissue samples by performing a method that includes obtaining a magnified image of a tissue sample. In an embodiment, the method further comprises generating an automatic segmentation of the tissue sample using a machine learning model. In an embodiment, the method further comprises providing the automatic segmentation to a user through a user interface. In an embodiment, the method further comprises obtaining modifications to the automatic segmentation through the user interface. In an embodiment, the method further comprises determining an edited segmentation from the modifications. In an embodiment, the method further comprises determining updated values of model parameters based on the edited segmentation.

### BRIEF DESCRIPTION OF THE DRAWINGS

FIG. 1 is a schematic of a state-of-the-art approach based on individual overlaying of information.

FIG. 2 is a schematic of an algorithm, in accordance with an embodiment of the present disclosure.

FIG. 3 is a schematic of a logical flow of visualization from a user interface (UI), in accordance with an embodiment of the present disclosure.

FIG. 4 is a schematic of a system, in accordance with an embodiment of the present disclosure.

FIG. 5 is a schematic of a logic flow, in accordance with an embodiment of the present disclosure.

FIG. 6 shows an example segmentation system, in accordance with an embodiment of the present disclosure.

FIG. 7 is an illustration of an example segmentation of a magnified image of a tissue sample, in accordance with an embodiment of the present disclosure.

FIG. 8 is a flow diagram of an example process for repeatedly training a machine learning model to segment magnified images of tissue samples, in accordance with an embodiment of the present disclosure.

FIG. 9 is a flow diagram of an example process for determining an expertise score that characterizes the predicted skill of a user in reviewing and editing segmentations of target tissue classes in magnified images of tissue samples, in accordance with an embodiment of the present disclosure.

FIG. 10 illustrates a block diagram of an exemplary computer system, in accordance with an embodiment of the present disclosure.

### DETAILED DESCRIPTION

Methods and systems for visualizing information on gigapixels Whole Slide Image are described. In the following description, numerous specific details are set forth in order to provide a thorough understanding of embodiments of the present disclosure. It will be apparent to one skilled in the art that embodiments of the present disclosure may be practiced without these specific details. In other instances, well-known aspects are not described in detail in order to not unnecessarily obscure embodiments of the present disclosure. Furthermore, it is to be understood that the various embodiments shown in the Figures are illustrative representations and are not necessarily drawn to scale.

One or more embodiments are directed to methods and systems for visualizing information on gigapixels Whole Slide Image (WSI). Embodiments may be directed to one or more of whole-slide images, deep zoom viewer, and/or results visualization.

Implementation of embodiments described herein may be helpful in visualizing information from multiple sources on a single whole slide image. An artificial intelligence (AI) algorithm produces many results/mask/information while analyzing whole slide images of biopsy samples. The visualization of such results can be helpful in maintaining the ability to explain results generated from AI algorithms.

To provide context, at present, to visualize results from an algorithm, a user has to select an image and result mask to visualize. The process has to be repeated for viewing results/information from multiple sources. By contrast, one or more embodiments described herein provide for unified visualization that dynamically picks information sources.

Embodiments disclosed herein can be implemented to reduce a doctor's efforts in analyzing results/reports generated by algorithms. Also, embodiments can be implemented to provide a deep learning algorithm as explainable AI algorithms.

To provide further context, existing visualization method are based on 1-to-1 mapping between an image and its mask. If there are N-result masks, then a doctor has to load N times one by one to analyze the result. By contrast, in embodiments described herein, a doctor need only load an image and its mask once. The algorithm dynamically picks a mask (out of N) which should be displayed at a current zoom factor of interest to the doctor.

Embodiments described herein can include a robust WSI viewer, a source of information to visualize, an algorithm to render a compatible visualization and unified visualization algorithm to select the source of information based on image viewer zoom factor. In one embodiment, the viewer is a standalone desktop application or cloud-enabled web application.

In accordance with an embodiment of the present disclosure, a unified visualization algorithm intelligently finds the zoom factor of visualization and chooses the best suitable information to be visualized. This approach can enable a doctor to work with various sources of information without an individual selection of such information one by one. For example, in histopathology, an AI/Deep Learning algorithm generates many information masks such as region-wise tumor mask and normal mask, region-wise score percentage in Immunohistochemistry report, cell marking, etc. An algorithm described herein dynamically picks suitable masks. As such, a doctor need not be concerned about which mask should be loaded in the viewer.

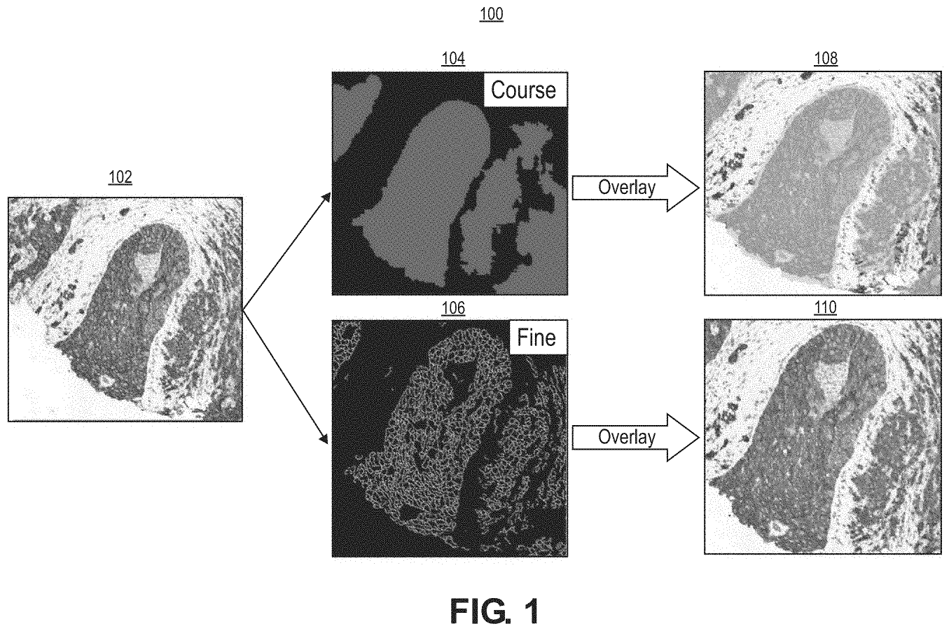

FIG. 1 is a schematic of a state-of-the-art approach based on individual overlaying of information.

Referring to FIG. 1, a process 100 begins at operation 102 with a patch from an input image. At operation 104, a first mask (information source 1, e.g., Coarse) is provided. At operation 106, a second mask (information source 2, e.g., Fine) such as Fine is provided. At operation 108, an overlay of Coarse is provided. At operation 110, an overlay of Fine is provided. The two overlay operations are distinct from one another.

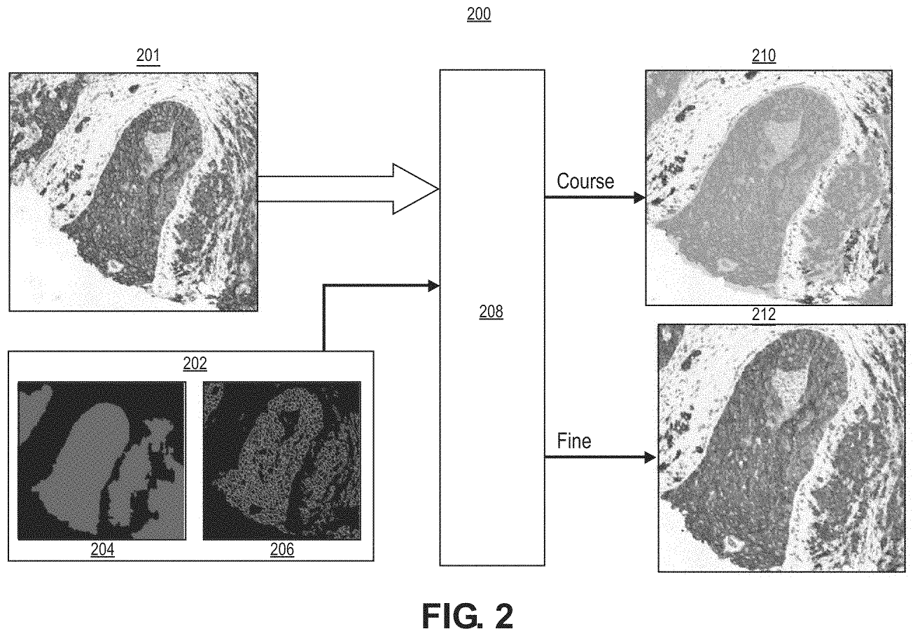

FIG. 2 is a schematic of an algorithm, in accordance with an embodiment of the present disclosure.

Referring to FIG. 2, a process 200 begins at operation 201 with a patch from an input image. At operation 202, a first mask 204 (information source 1) and a second mask 206 (information source 2) are provided as an all information source. At operation 208, an algorithm choosing information source is used based on Zoom level. A Coarse image 210 and/or a Fine image 212 can then be provided.

To provide further context, Digital Pathology has recently gained significant traction for applications in telemedicine and machine learning-based slide analysis. Typically, a WSI has 3 channels (RGB) with the size of few gigabytes and dimensions of 100K×200K pixels. These images are based on a pyramidal image with various zoom factors of multiples of 2. There can be a need for a separate viewer to visualize these images in a web application or desktop application since images cannot be viewed in normal image viewers. To highlight a specific finding on such images, there may be a need for the separate mask (termed as a source of information) and load in viewer for visualization. However, when there is a need to visualize multiple findings from different sources then challenges can arise such as a need to create separate masks and superimposition of one after another to analyze findings. The use of separate mask images can create a problem of switching one after another. This can lead to loss of focus from one finding to another. To address such issues, in one or more embodiments described herein, a unified visualization algorithm is implemented where the algorithm dynamically selects information/mask to visualize based on zoom factor (i.e., viewing Coarse to Fine details of tissue).

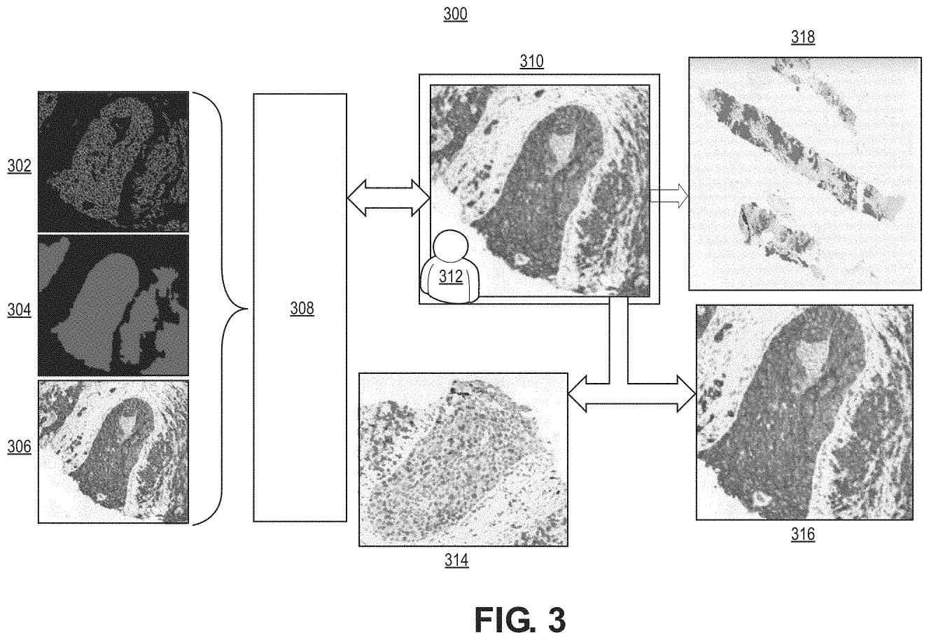

FIG. 3 is a schematic of a logical flow 300 of visualization from a user interface (UI), in accordance with an embodiment of the present disclosure.

Referring to FIG. 3, input masks 302, 304 and 306 are provided. At operation 308, an algorithm is used which selects a mask (Source of information) based on user input. A user 312 makes a request for specific information to display on an image viewer 310. Exemplary images include Fine-Nuclei 314, Fine-Membrane 316, and/or Coarse 318.

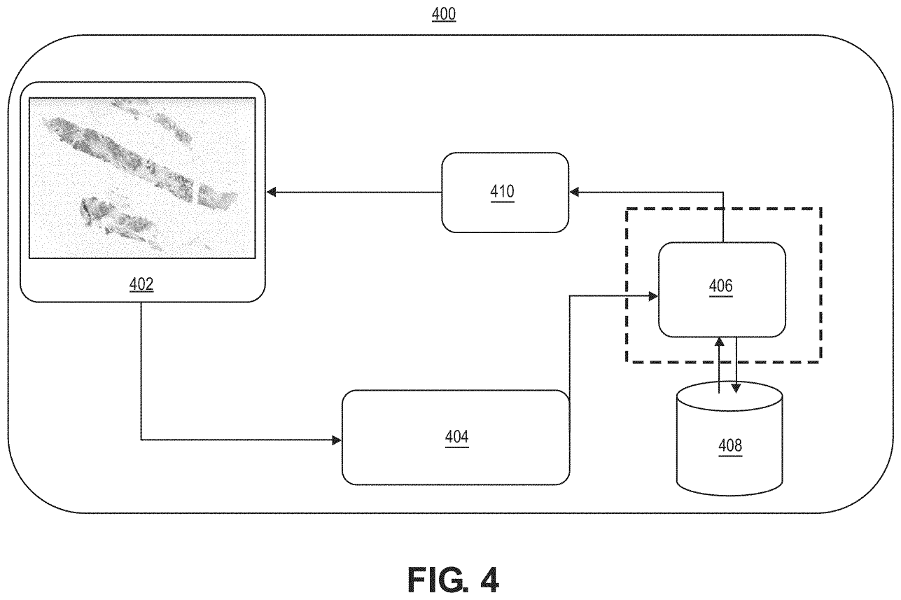

FIG. 4 is a schematic of a system, in accordance with an embodiment of the present disclosure.

Referring to FIG. 4, a system 400 includes a WSI viewer 402. At operation 404, a user requests to visualize information from other sources on a same image. At operation 406, an algorithm dynamically estimates zoom level which may include interaction with or use of a slide/mask in a database 408. A multi-source information image is provided at 410.

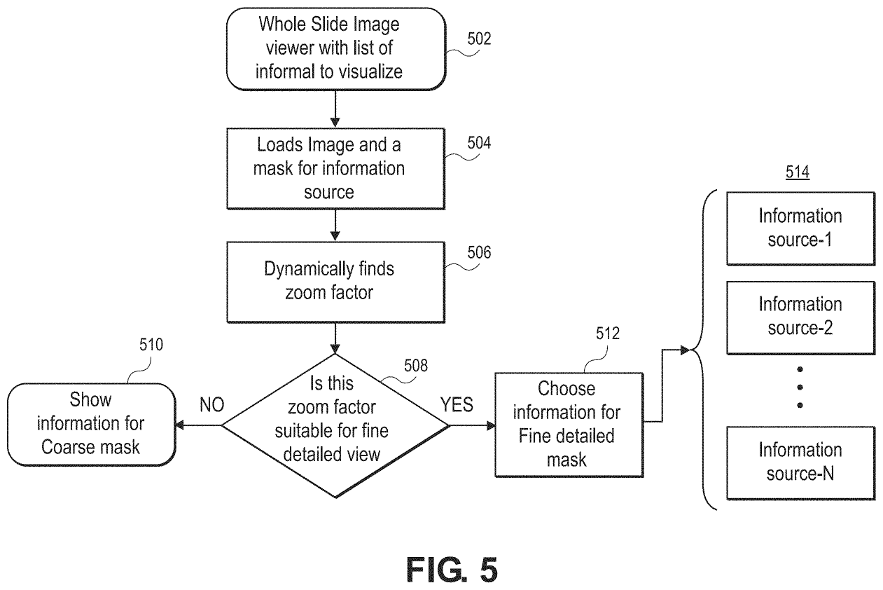

FIG. 5 is a schematic of a logic flow 500, in accordance with an embodiment of the present disclosure.

Referring to FIG. 5, at operation 502, a Whole Slide Image viewer with a list of information to visualize is provided. At operation 504, an image and a mask are loaded for an information source. At operation 506, the flow dynamically finds a zoom factor. At operation 508, a query is made: Is this zoom factor suitable for fine detailed view?” If no, then information for a Coarse mask is shown at operation 510. If yes, then information for a Fine detailed mask is chosen at operation 512, e.g., based on 514: Information source-1 . . . Information source-N.

In a particular embodiment, the information is visualized in a single-screen view. In another particular embodiment, the information is visualized in a multi-screen view. In a latter such embodiment, a four-stain viewer is used.

In accordance with one or more embodiments of the present disclosure, a unified visualization algorithm intelligently finds the zoom factor of visualization and chooses the best suitable information to be visualized. For example, in histopathology, viewing tumorous breast tissue can be used to visualize tumorous cells. The algorithmic visualization helps to maintain a solution as an explainable AI. This assists a doctor to work with various sources in information without an individual selection of such information one by one. For example, in histopathology, AI/Deep Learning algorithms generate many information masks such as region-wise tumor/normal, region-wise score percentage (IHC (Immunohistochemistry) report), cell marking.

In another aspect, interactive training of a machine learning model for tissue segmentation is described.

This specification relates to processing magnified images of tissue samples using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output. This specification describes a system implemented as computer programs on one or more computers in one or more locations for segmenting magnified images of tissue samples into respective tissue classes.

Particular embodiments of the subject matter described in this specification can be implemented so as to realize one or more of the following advantages. The segmentation system described in this specification enables a user (e.g., a pathologist) to work in tandem with a machine learning model to segment magnified images of tissue samples into (target) tissue classes in a manner that is both time-efficient and highly accurate. This specification describes techniques for computing an “expertise” score for a user that characterizes the predicted skill of the user in manually reviewing and editing segmentations (i.e., of the target tissue classes). The expertise scores can be used to improve the performance of the segmentation system. For example, the expertise scores can be used to improve the quality of the training data used to train the segmentation system, e.g., by determining whether to include a segmentation generated by a user in the training data based on the expertise score of the user.

This specification describes a segmentation system for segmenting magnified images of tissue samples (e.g., that are generated using a microscope, e.g., an optical microscope) into respective tissue classes. More specifically, the segmentation system can process a magnified image of a tissue sample to identify a respective (target) tissue class corresponding to each pixel of the image. The (target) tissue class of a pixel in the image characterizes the type of tissue in the portion of the tissue sample corresponding to the pixel.

As used throughout this document, a “microscope” can refer to any system that can generate magnified images of a sample, e.g., using a 1-D array of photodetectors, or using a 2-D array of charge-coupled devices (CCDs).

The segmentation system can be configured to segment images into any appropriate set of tissue classes. In one example, the segmentation system may segment images into cancerous tissue and non-cancerous tissue. In another example, the segmentation system may segment images into: healthy tissue, cancerous tissue, and necrotic tissue. In another example, the segmentation system may segment images into: muscle tissue, nervous tissue, connective tissue, epithelial tissue, and “other” tissue. The segmentation system can be used in any of a variety of settings, e.g., to segment magnified images of tissue samples that are obtained from patients through biopsy procedures. The tissue samples can be samples of any appropriate sort of tissue, e.g., prostate tissue, breast tissue, liver tissue, or kidney tissue. The segmentations generated by the segmentation system can be used for any of a variety of purposes, e.g., to characterize the presence or extent of disease (e.g., cancer).

Manually segmenting a single magnified image of a tissue sample may be a challenging task that consumes hours of time, e.g., as a result of the high-dimensionality of the image, which can have on the order of 10¹⁰ pixels. On the other hand, a machine learning model can be trained to automatically segment magnified images of tissue samples in considerably less time (e.g., in seconds or minutes, e.g., 10-30 minutes). However, it may be difficult to train a machine learning model to achieve a level of accuracy that would be considered acceptable for certain practical applications, e.g., identifying cancerous tissue in biopsy samples. In particular, the microscopic appearance of tissue can be highly complex and variable due to factors that are both intrinsic to the tissue (e.g., the type and stage of the disease present in tissue) and extrinsic to the tissue (e.g., how the microscope is calibrated and the procedure used to stain the tissue). This makes it hard to aggregate a set of labeled training data (i.e., for training a machine learning model) that is sufficiently large to capture the full scope of possible variations in the microscopic appearance of tissue.

The segmentation system described in this specification enables a user (e.g., a pathologist) to work in tandem with a machine learning model to segment tissue samples in a manner that is both time-efficient and highly accurate. To segment an image, the machine learning model first generates an automatic segmentation of the image which is subsequently provided to the user through a user interface that enables the user to review and manually edit the automatic segmentation as necessary. The “edited” segmentation is provided by the segmentation system as an output, and is also used to update the parameter values of the machine learning model (e.g., immediately or at a subsequent time point) to cause it to generate segmentations that more closely match those of the user.

In this manner, rather than being trained once on a static and limited set of training data (as in some conventional systems), the machine learning model continually learns and adapts its parameter values based on the feedback being provided by the user through the edited segmentations. Moreover, rather than being required to segment an image from scratch, the user can start from the automatic segmentation generated by the machine learning model, and may be required to make fewer corrections to the automatic segmentations over time as the machine learning model continually improves.

The term “tissue” here refers to a group of cells of similar structure and function, as opposed to individual cells. The color, texturing, and similar image properties of tissues are significantly different from those of individual cells, so image processing techniques applicable to cell classification often are not applicable to segmenting images of tissue samples and classifying those segments.

These features and other features are described in more detail below.

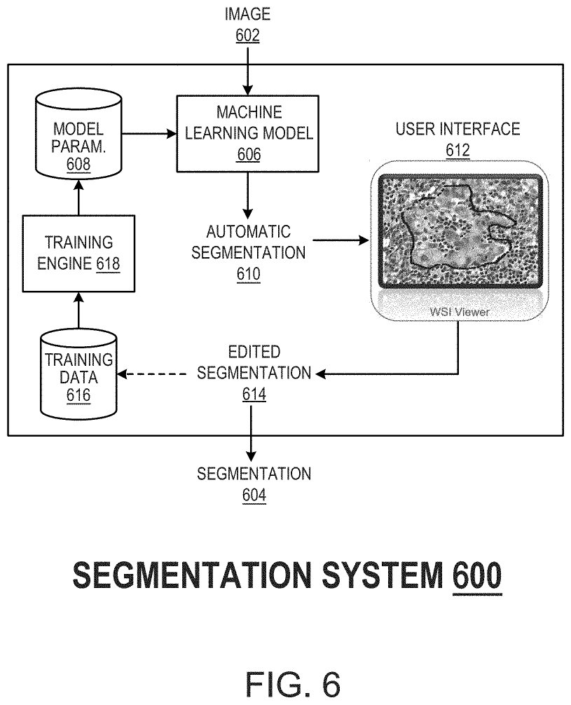

FIG. 6 shows an example segmentation system 600. The segmentation system 600 is an example of a system implemented as computer programs on one or more computers in one or more locations in which the systems, components, and techniques described below are implemented.

The segmentation system 600 is configured to process a magnified image 602 of a tissue sample to generate a segmentation 604 of the image 602 into respective tissue classes, e.g., cancerous and non-cancerous tissue classes.

The image 602 may be, e.g., a whole slide image (WSI) of a tissue sample mounted on a microscope slide, where the WSI is generated using an optical microscope and captured using a digital camera. The image 602 can be represented in any of a variety of ways, e.g., as a two-dimensional (2-D) array of pixels, where each pixel is associated with a vector of numerical values characterizing the appearance of the pixel, e.g., a 3-D vector defining the red-green-blue (RGB) color of the pixel. The array of pixels representing the image 602 may have a dimensionality on the order of, e.g., 10⁵×10⁵ pixels, and may occupy several gigabytes (GB) of memory. The system 600 may receive the image 602 in any of a variety of ways, e.g., as an upload from a user of the system using a user interface made available by the system 600.

The machine learning model 606 is configured to process the image 602, features derived from the image 602, or both, in accordance with current values of a set of model parameters 608 to generate an automatic segmentation 610 of the image 602 that specifies a respective tissue class corresponding to each pixel of the image 602. The machine learning model 606 may be, e.g., a neural network model, a random forest model, a support vector machine model, or a linear model. In one example, the machine learning model may be a convolutional neural network having an input layer that receives the image 602, a set of convolutional layers that process the image to generate alternative representations of the image at progressively higher levels of abstraction, and a soft-max output layer. In another example, the machine learning model may be a random forest model that is configured to process a respective feature representation of each pixel of the image 602 to generate an output that specifies a tissue class for the pixel. In this example, a feature representation of a pixel refers to an ordered collection of numerical values (e.g., a vector of numerical values) that characterizes the appearance of the pixel. The feature representation may be generated using, e.g., histogram of oriented gradient (HOG) features, speeded up robust features (SURF), or scale-invariant feature transform (SIFT) features.

The model parameters 608 are a collection of numerical values that are learned during training of the machine learning model 606 and which specify the operations performed by the machine learning model 606 to generate an automatic segmentation 610 of the image 602. For example, if the machine learning model 606 is a neural network, the model parameters 608 may specify the weight values of each layer of the neural network, e.g., the weight values of the convolutional filters of each convolutional layer of the neural network. (The weight values for a given layer of the neural network may refer to the values associated with the connections between neurons of the given layer and neurons in the preceding layer of the neural network). As another example, if the machine learning model 606 is a random forest model, the model parameters 608 may specify the parameter values of the respective splitting function used at each node of each decision tree of the random forest. As another example, if the machine learning model 606 is a linear model, the model parameters 608 may specify the coefficients of the linear model.

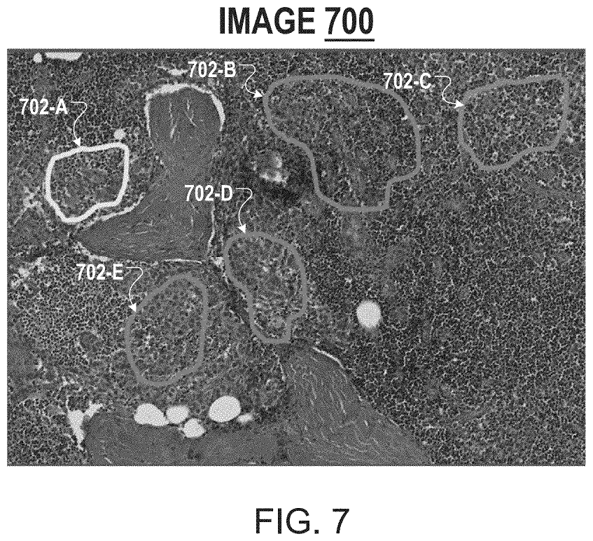

The system 600 displays the image 602 and the automatic segmentation 610 of the image on a display device of a user interface 612. For example, the system 600 may display a visualization that depicts the automatic segmentation 610 overlaid onto the image 602, as illustrated with reference to FIG. 7. The user interface 612 may have any appropriate sort of display device, e.g., a liquid-crystal display (LCD).

The user interface 612 enables a user of the system (e.g., a pathologist) to view the image 602 and the automatic segmentation 610, and to edit the automatic segmentation 610 as necessary by specifying one or more modifications to the automatic segmentation 610. Modifying the automatic segmentation 610 refers to changing the tissue class specified by the automatic segmentation 610 to a different tissue class for one or more pixels of the image 602. Generally, the user may edit the automatic segmentation 610 to correct any errors in the automatic segmentation 610. For example, the user interface 612 may enable the user to “deselect” a region of the image that is specified by the automatic segmentation as having a certain tissue class (e.g., cancerous tissue) by re-labeling the region as having a default tissue class (e.g., non-cancerous tissue). As another example, the user interface 612 may enable the user to “select” a region of the image and label the region as having a particular tissue class (e.g., cancerous tissue). As another example, the user interface 612 may enable the user to change the region of the image labelled as having a particular tissue class. The change in a region can be performed, e.g., by dragging corners of a polygon surrounding the region.

The user may interact with the user interface 612 to edit the automatic segmentation 610 in any of a variety of ways, e.g., using a computer mouse, a touch screen, or both. For example, to select a region of the image and label the region as having a tissue class, the user may use a cursor to draw a closed loop around the region of the image, and then select the desired tissue class from a drop down menu. The user may indicate that editing of the automatic segmentation is complete by providing an appropriate input to the user interface (e.g., clicking a “Finish” button), at which point the edited segmentation 614 (i.e., that has been reviewed and potentially modified by the user) is provided as an output. For example, the output segmentation 604 may be stored in a medical records data store in association with a patient identifier.

In addition to providing the edited segmentation 614 as an output, the system 600 may also use the edited segmentation 614 to generate a training example that specifies: (i) the image, and (ii) the edited segmentation 614, and store the training example in a set of training data 616. Generally, the training data 616 stores multiple training examples (i.e., that each specify a respective image and an edited segmentation), and may be continually augmented over time as users generate edited segmentations of new images. The system 600 uses a training engine 618 to repeatedly train the machine learning model 606 on the training data 616 by updating the model parameters 608 to encourage the machine learning model 606 to generate automatic segmentations that match the edited segmentations specified by the training data 616.

The training engine 618 may train the machine learning model 606 on the training data 616 whenever a training criterion is satisfied. For example, the training engine 618 may train the machine learning model 606 each time a predefined number of new training examples are added to the training data 616. As another example, the training engine 618 may train the machine learning model 606 each time the machine learning model 606 generates an automatic segmentation 610 that differs substantially from the corresponding edited segmentation 614 that is specified by the user. In this example, the training engine 618 may use the substantial difference between the automatic segmentation 610 and the edited segmentation 614 as a cue that the machine learning model 606 failed to correctly segment an image and should be trained to avoid repeating the errors. The training engine 618 may determine that two segmentations are substantially different if a similarity measure between the segmentations (e.g., a Jaccard index similarity measure) does not satisfy a predefined threshold.

The manner in which the training engine 618 trains the machine learning model 606 on the training data 616 depends on the form of the machine learning model 606. In some cases, the training engine 618 may train the machine learning model 606 by determining an adjustment to the current values of the model parameters 608. In other cases, the training engine 618 may start by initializing the model parameters 608 to default values each time the machine learning model 606 is trained, e.g., values that are sampled from a predefined probability distribution, e.g., a standard Normal distribution.

Take, as an example, a case where the machine learning model 606 is a neural network model, and the training engine 618 trains the neural network model using one or more iterations of stochastic gradient descent. In this example, at each iteration, the training engine 618 selects a “batch” (set) of training examples from the training data 616, e.g., by randomly selecting a predefined number of training examples. The training engine 618 processes the image 602 from each selected training example using the machine learning model 606 in accordance with the current values of the model parameters 608, to generate a corresponding automatic segmentation. The training engine 618 determines gradients of an objective function with respect to the model parameters 608, where the objective function measures a similarity between: (i) the automatic segmentations generated by the machine learning model 606, and (ii) the edited segmentations specified by the training examples. The training engine 618 then uses the gradients of the objective function to adjust the current values of the model parameters 608 of the machine learning model 606. The objective function may be, e.g., a pixel-wise cross-entropy objective function, the training engine 618 may determine the gradients using backpropagation techniques, and the training engine 618 may adjust the current values of the model parameters 608 using any appropriate gradient descent technique, e.g., Adam or RMSprop.

Optionally, the training engine 618 may preferentially train the machine learning model 606 on training examples that were generated more recently, i.e., rather than treating each training example equally. For example, the training engine 618 may train the machine learning model 606 on training examples that are sampled from the training data 616, where training examples that were generated more recently have a higher likelihood of being sampled than older training examples. Preferentially training the machine learning model 606 on training examples that were generated more recently can enable the machine learning model 606 to focus on learning from newer training examples while maintaining the insights gained from older training examples.

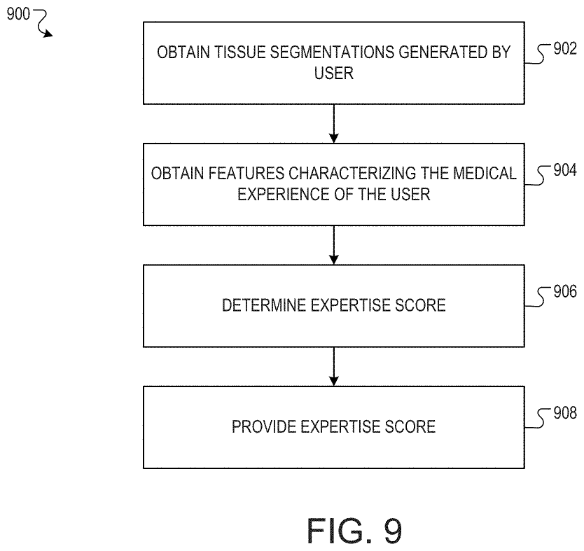

Generally, the system 600 trains the machine learning model 606 to generate automatic segmentations 610 that match edited segmentations 614 specified by users of the system 600, e.g., pathologists. However, certain users may be more skilled than others in reviewing and editing automatic segmentations generated by the machine learning model 606 for accuracy. For example, a more experienced pathologist may achieve a higher accuracy in reviewing and editing segmentations of complex and ambiguous tissue samples than a more junior pathologist. In some implementations, each user of the system 600 may be associated with an “expertise” score that characterizes the predicted skill of the user in reviewing and editing segmentations. In these implementations, the machine learning model 606 may be trained using only edited segmentations that are generated by users with a sufficiently high expertise score, e.g., an expertise score that satisfies a predetermined threshold. An example process for determining an expertise score for a user is described in more detail with reference to FIG. 9.

Determining whether to train the machine learning model 606 on an edited segmentation based on the expertise score of the user that generated the segmentation can improve the performance of the machine learning model 606 by improving the quality of the training data. Optionally, users of the system 600 may be compensated (e.g., financially or otherwise) for providing segmentations that are used to train the machine learning model 606. In one example, the amount of compensation provided to a user may depend on the expertise score of the user, and users with higher expertise scores may receive more compensation than users with lower expertise scores.

Optionally, the system 600 may be a distributed system where various components of the system are implemented remotely from one another and communicate over a data communication network, e.g., the Internet. For example, the user interface 612 (including the display device) may be implemented in a clinical environment (e.g., a hospital), while the machine learning model 606 and the training engine 618 may be implemented in a remote data center.

Optionally, a user of the system 600 may be provided the option of disabling the machine learning model 606. If this option is selected, the user can load images 602 and manually segment them without use of the machine learning model 606.

FIG. 7 is an illustration of a magnified image 700 of a tissue sample, where the regions 702-A-E (and the portion of the image outside of the regions 702-A-E) correspond to respective tissue classes.

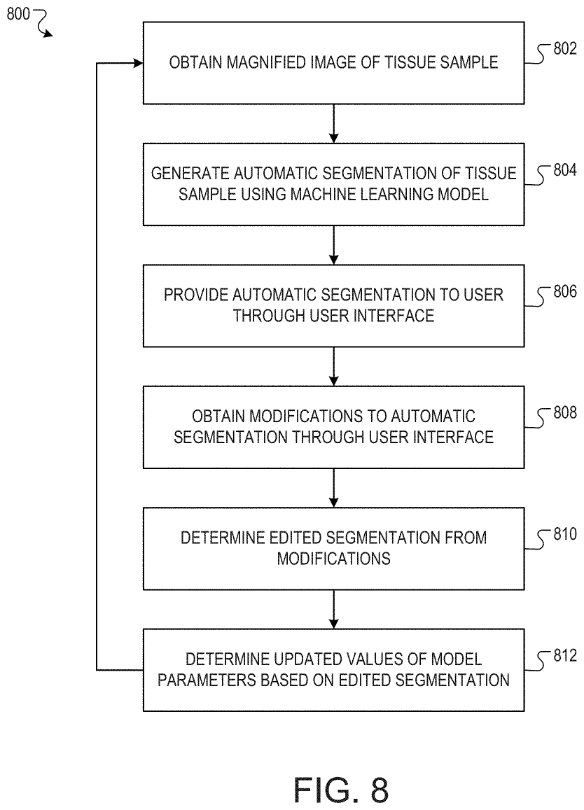

FIG. 8 is a flow diagram of an example process 800 for repeatedly training a machine learning model to segment magnified images of tissue samples. For convenience, the process 800 will be described as being performed by a system of one or more computers located in one or more locations. For example, a segmentation system, e.g., the segmentation system 600 of FIG. 6, appropriately programmed in accordance with this specification, can perform the process 800.

The system obtains a magnified image of a tissue sample (802). For example, the image may be a magnified whole slide image of a biopsy sample from a patient that is generated using a microscope.

The system processes an input including: (i) the image, (ii) features derived from the image, or (iii) both, in accordance with current values of the model parameters of the machine learning model to generate an automatic segmentation of the image into a set of (target) tissue classes (804). The automatic segmentation specifies a respective tissue class corresponding to each pixel of the image. The tissue classes may include cancerous tissue and non-cancerous tissue. The machine learning model may be a neural network model, e.g., a convolutional neural network model with one or more convolutional layers.

The system provides an indication of: (i) the image, and (ii) the automatic segmentation of the image, to the user through a user interface (806). For example, the system may provide a visualization that depicts the automatic segmentation overlaid on the image through a display device of the user interface. The visualization of the automatic segmentation overlaid on the image may indicate the predicted tissue type of each of the regions delineated by the automatic segmentation. For example, the visualization may indicate the predicted tissue type of a region by colorizing the region based on the tissue type, e.g., cancerous tissue is colored red, while non-cancerous tissue is colored green.

The system obtains an input specifying one or more modifications to the automatic segmentation of the image from the user through the user interface (808). Each modification to the automatic segmentation may indicate, for one or more pixels of the image, a change to the respective tissue class specified for the pixel by the automatic segmentation.

The system determines an edited segmentation of the image (810). For example, the system may determine the edited segmentation of the image by applying the modifications specified by the user through the user interface to the automatic segmentation of the image.

The system determines updated values of the model parameters of the machine learning model based on the edited segmentation of the image (812). For example, the system may determine gradients of an objective function that characterizes a similarity between: (i) the automatic segmentation of the image, and (ii) the edited segmentation of the image, and then adjust the values of the model parameters using the gradients. In some cases, the system may determine updated values of the model parameters of the machine learning model only in response to determining that a training criterion is satisfied, e.g., that a predefined number of new edited segmentations have been generated since the last time the model parameters were updated. After determining updated values of the model parameters, the system may return to step 802. If the training criterion is not satisfied, the system may return to step 802 without training the machine learning model.

FIG. 9 is a flow diagram of an example process 900 for determining an expertise score that characterizes the predicted skill of a user in reviewing and editing segmentations of magnified images of tissue samples. For convenience, the process 900 will be described as being performed by a system of one or more computers located in one or more locations. For example, a segmentation system, e.g., the segmentation system 600 of FIG. 6, appropriately programmed in accordance with this specification, can perform the process 900.

The system obtains one or more tissue segmentations that were generated by the user (902). Each tissue segmentation corresponds to a magnified image of a tissue sample and specifies a respective tissue class for each pixel of the image. In some implementations, the user may have performed the segmentations from scratch, e.g., without the benefit of starting from automatic segmentations generated by a machine learning model.

The system obtains one or more features characterizing the medical experience of the user, e.g., in the field of pathology (904). For example, the system may obtain features characterizing one or more of: the number of years of experience of the user in the field of pathology, the number of academic publications of the user in the field of pathology, the number of citations of the academic publications of the user in the field of pathology, the academic performance of the user (e.g., in medical school), and the position currently held by the user (e.g., attending physician).

The system determines the expertise score for the user based on: (i) the tissue segmentations generated by the user, and (ii) the features characterizing the medical experience of the user (906). For example, the system may determine the expertise score as a function (e.g., a linear function) of: (i) a similarity measure between the segmentations generated by the user and corresponding “gold standard” segmentations of the same images, and (ii) the features characterizing the medical experience of the user. A gold standard segmentation of an image may be a segmentation that is generated by a user (e.g., a pathologist) that is recognized as having a high level of expertise in performing tissue segmentations. A similarity measure between two segmentations of an image can be evaluated using, e.g., a Jaccard index. The expertise score for a user may be represented as a numerical value, e.g., in the range [0,1].

The system provides the expertise score for the user (908). For example, the system may provide the expertise score for the user for use in determining whether segmentations generated by the user should be included in training data used to train a machine learning model to perform automatic tissue sample segmentations. In this example, segmentations generated by a user may be included in the training data only if, e.g., the expertise score for the user satisfies a threshold. In another example, the system may provide the expertise score for the user for use in determining how the user should be compensated (e.g., financially or otherwise) for providing tissue sample segmentations, e.g., where having a higher expertise score may result in higher compensation.

This specification uses the term “configured” in connection with systems and computer program components. For a system of one or more computers to be configured to perform particular operations or actions means that the system has installed thereon software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor (e.g., central processing unit (CPU), graphics processing unit (GPU)), a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any disclosure or on the scope of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially be claimed as such, one or more features from a claimed combination can in some cases be excised from the combination, and the claimed combination may be directed to a sub-combination or variation of a sub-combination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Embodiments of the present disclosure may be provided as a computer program product, or software, that may include a machine-readable medium having stored thereon instructions, which may be used to program a computer system (or other electronic devices) to perform a process according to embodiments of the present disclosure. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical or other form of propagated signals (e.g., infrared signals, digital signals, etc.)), etc.

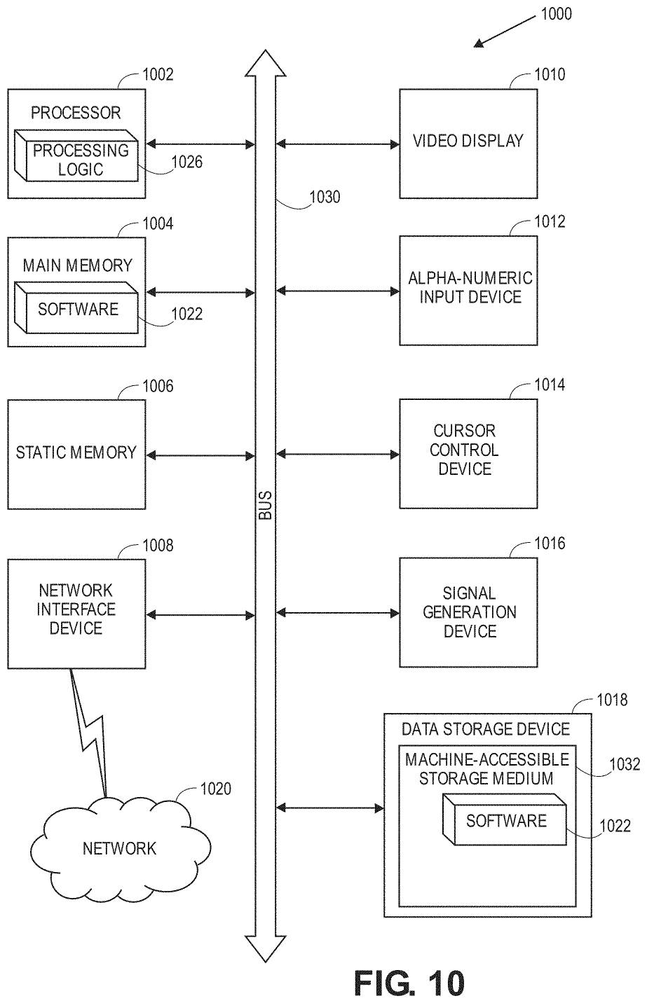

FIG. 10 illustrates a diagrammatic representation of a machine in the exemplary form of a computer system 1000 within which a set of instructions, for causing the machine to perform any one or more of the methodologies described herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies described herein.

The exemplary computer system 1000 includes a processor 1002, a main memory 1004 (e.g., read-only memory (ROM), flash memory, dynamic random access memory (DRAM) such as synchronous DRAM (SDRAM) or Rambus DRAM (RDRAM), etc.), a static memory 1006 (e.g., flash memory, static random access memory (SRAM), etc.), and a secondary memory 1018 (e.g., a data storage device), which communicate with each other via a bus 1030.

Processor 1002 represents one or more general-purpose processing devices such as a microprocessor, central processing unit, or the like. More particularly, the processor 1002 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 1002 may also be one or more special-purpose processing devices such as an application specific integrated circuit (ASIC), a field programmable gate array (FPGA), a digital signal processor (DSP), network processor, or the like. Processor 1002 is configured to execute the processing logic 1026 for performing the operations described herein.

The computer system 1000 may further include a network interface device 1008. The computer system 1000 also may include a video display unit 1010 (e.g., a liquid crystal display (LCD), a light emitting diode display (LED), or a cathode ray tube (CRT)), an alphanumeric input device 1012 (e.g., a keyboard), a cursor control device 1014 (e.g., a mouse), and a signal generation device 1016 (e.g., a speaker).

The secondary memory 1018 may include a machine-accessible storage medium (or more specifically a computer-readable storage medium) 1032 on which is stored one or more sets of instructions (e.g., software 1022) embodying any one or more of the methodologies or functions described herein. The software 1022 may also reside, completely or at least partially, within the main memory 1004 and/or within the processor 1002 during execution thereof by the computer system 1000, the main memory 1004 and the processor 1002 also constituting machine-readable storage media. The software 1022 may further be transmitted or received over a network 1020 via the network interface device 1008.

While the machine-accessible storage medium 1032 is shown in an exemplary embodiment to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, and optical and magnetic media.

Thus, methods and systems for visualizing information on gigapixels Whole Slide Image (WSI) have been disclosed.

What is claimed is:

1. A method for visualizing information, the method comprising:
providing an image viewer with a list of information to visualize;
loading an image and a mask for an information source;
dynamically finding a zoom factor;
if the zoom factor is not suitable for fine detailed view, then show information for a coarse mask; or
if the zoom factor is suitable for fine detailed view, then choose information for a fine detailed mask from a plurality of information sources.

2. The method of claim 1, wherein the information is visualized in a multi-screen view.

3. The method of claim 2, wherein the multi-screen view is within a single display apparatus.

4. The method of claim 2, wherein the multi-screen view is over two or more display apparatuses.

5. The method of claim 1, further comprising:
determining a control parameter that dictates an opacity of the mask.

6. The method of claim 1, further comprising:
obtaining a user driven threshold value which controls the area of the mask.

7. The method of claim 1, further comprising:
determining a control parameter that dictates an opacity of the mask; and
obtaining a user driven threshold value which controls the area of the mask.

8. A method for repeatedly training a machine learning model to segment magnified images of tissue samples, comprising:
obtaining a magnified image of a tissue sample;
generating an automatic segmentation of the tissue sample using a machine learning model;
providing the automatic segmentation to a user through a user interface;
obtaining modifications to the automatic segmentation through the user interface;
determining an edited segmentation from the modifications; and
determining updated values of model parameters based on the edited segmentation.

9. The method of claim 8, further comprising:
repeating the process with the updated values of model parameters.

10. The method of claim 8, wherein determining updated values of model parameters is executed when a threshold value is reached.

11. The method of claim 10, wherein the threshold value is a the formation of a preset number of edited segmentations.

12. The method of claim 10, wherein the threshold value is a user expertise score of the user that is above a certain value.

13. The method of claim 12, wherein the user expertise score is formed by a method comprising:
obtaining tissue segments generated by the user;
comparing the tissue segments to gold standard tissue segments;
obtaining features characterizing the medical experience of the user;
determining the expertise score based on the comparison to the gold standard tissue segments and the features characterizing the medical experience of the user.

14. The method of claim 13, wherein features characterizing the medical experience of the user includes one or more of, medical school performance, years in a certain medical field, position title, number of articles written, and citations from other articles.

15. The method of claim 13, wherein the gold standard tissue segments are generated by a well-respected user in a given medical field.

16. The method of claim 8, wherein the segmentation refers to classifying different areas tissue sample as different tissue types, wherein the different tissue types includes one or more of cancerous tissue, healthy tissue, and necrotic tissue.

17. The method of claim 8, wherein the user interface comprises a display apparatus and an input device, wherein the input device comprises a touch screen and/or a mouse.

18. A non-transitory computer readable storage medium having data stored representing software executable by a computer, the software including instructions for repeatedly training a machine learning model to segment magnified images of tissue samples by performing a method comprising:
obtaining a magnified image of a tissue sample;
generating an automatic segmentation of the tissue sample using a machine learning model;
providing the automatic segmentation to a user through a user interface;
obtaining modifications to the automatic segmentation through the user interface;
determining an edited segmentation from the modifications; and
determining updated values of model parameters based on the edited segmentation.

19. The non-transitory computer readable storage medium of claim 18, further comprising:
repeating the process with the updated values of model parameters.

20. The non-transitory computer readable storage medium of claim 18, wherein determining updated values of model parameters is executed when a threshold value is reached.
